(19) United States
(12) Patent Application Publication
(10) Pub. No.: US 2022/0226386 A1
(43) Pub. Date: Jul. 21, 2022

(54) FUNCTIONAL RECOVERY FROM CEREBRAL INFARCTION

(71) Applicant: Mesoblast International Sárl, Meyrin (CH)

(72) Inventor: Silviu ITESCU, Melbourne (AU)

(21) Appl. No.: 17/612,939

(22) PCT Filed: May 22, 2020
(86) PCT No.: PCT/EP2020/064271

§ 371 Date: Nov. 19, 2021

(30) Foreign Application Priority Data
May 23, 2019 (AU) 2019901756
Jun. 11, 2019 (AU) 2019902024

(51) Int. Cl. A61K 35/28; A61P 9/10; C12N 5/0775

(52) U.S. Cl. CPC A61K 35/28; C12N 5/0662; A61P 9/10

(57) ABSTRACT

The present disclosure provides methods of treating a subject who has suffered a cerebral infarction, the method comprising administering systemically to the subject a population of cells enriched for mesenchymal lineage precursor or stem cells (MLPSCs) such as STRO-1⁺ cells or progeny thereof to increase stimulus-induced cortical activation or reduce infarct volume.

### FIELD

The present disclosure relates to methods of treating cerebral infarction in a human subject.

### BACKGROUND

Cerebral infarction remains a key cause of morbidity and mortality in the industrialized world. Cerebral infarction is the third leading cause of mortality. Cerebral infarction is the rapidly developing loss of brain function(s) due to disturbance in the blood supply to the brain. There are two common types of cerebral infarction: (i) ischemic cerebral infarction, which is caused by a temporary or permanent occlusion to blood flow to the brain, and accounts for 85% of cerebral infarction cases, and (ii) hemorrhagic cerebral infarction, which is caused by a ruptured blood vessel and accounts for the majority of the remaining cases. Cerebral infarction often results in neuronal cell death and can lead to death. The most common cause of ischemic cerebral infarction is occlusion of the middle cerebral artery (the intra-cranial artery downstream from the internal carotid artery), which damages cerebrum (e.g., cerebral cortex), e.g., the motor and sensory cortices of the brain. Such damage results in hemiplegia, hemi-anesthesia and, depending on the cerebral hemisphere damaged, either language or visuo-spatial deficits. The affected volume of brain and its compromised function can be visualised by functional imaging techniques such as Blood Oxygenation Level Dependent (BOLD) magnetic resonance imaging (MRI), which image accompanying reductions in blood flow in the affected brain region(s).

Cerebral infarction can affect subjects physically, mentally, emotionally, or a combination of the three.

Some of the physical disabilities that can result from cerebral infarction include muscle weakness, numbness, pressure sores, pneumonia, incontinence, apraxia (inability to perform learned movements), difficulties carrying out daily activities, appetite loss, speech loss, vision loss, and pain. If the cerebral infarction is severe enough, or in a certain location such as parts of the brainstem, coma or death can result.

Emotional problems resulting from cerebral infarction can result from direct damage to emotional centers in the brain or from frustration and difficulty adapting to new limitations. Post-cerebral infarction emotional difficulties include depression, anxiety, panic attacks, flat affect (failure to express emotions), mania, apathy, and psychosis.

Cognitive deficits resulting from cerebral infarction include perceptual disorders, speech problems, dementia, and problems with attention and memory. A cerebral infarction sufferer may be unaware of his or her own disabilities, a condition called anosognosia. In a condition called hemispatial neglect, a patient is unable to attend to anything on the side of space opposite to the damaged hemisphere.

There are no approved therapies for cerebral infarction except tissue plasminogen activator (TPA) if administered within three hours of presentation of symptom onset. Given the lack of therapeutic options for the treatment of cerebral infarction there is a strong need for additional therapies that promote reperfusion, or are neuroprotective.

### SUMMARY

The present disclosure is based on the inventors'surprising finding that systemic administration of human mesenchymal lineage precursor or stem cells (MLPSCs), e.g., STRO-1⁺ human mesenchymal precursor cells (hMPCs) results in improved functional recovery within a cortical volume affected by an infarct, as assessed by functional imaging.

Accordingly, in a first aspect described herein is a method for increasing cortical activation or reducing infarct volume following a cerebral infarction, the method comprising systemic administration of a therapeutically effective amount of a human cell population enriched for mesenchymal lineage precursor or stem cells (MLPSCs) to a human subject in need thereof.

In some embodiments the cerebral infarction is an ischemic cerebral infarction. In some embodiments, where the cerebral infarction is an ischemic cerebral infarction, the cerebral infarction in the subject to be treated was caused by hypoxic ischemic encephalopathy (HIE). In other embodiments the cerebral infarction is a hemorrhagic cerebral infarction.

In some embodiments the cerebral infarction is in motor cortex. In some embodiments the affected volume is reduced following the administration. In some embodiments cortical activation is increased. In some embodiments motor function is improved in the human subject. In some embodiments increased cortical activation following treatment is in response to contralateral tactile stimulation. In some embodiments the cortical activation is increased within the volume of the infarct.

In some embodiments systemic administration of the human cell population is performed at about 24 hours or less following the cerebral infarction. In other embodiments the systemic administration is performed at about 12 hours or less following the cerebral infarction.

In some embodiments the MLPSCs are STRO-1⁺ MPCs. In some embodiments the STRO-1⁺ MPCs are STRO-1^{bright} MPCs. In some embodiments the STRO-1⁺ MPCs are tissue non-specific alkaline phosphatase (TNAP)⁺ or CD146⁺.

In other embodiments the MLPSCs are mesenchymal stem cells.

In some embodiments the human cell population to be administered is an allogeneic human cell population. In other embodiments the human cell population is an autogeneic human cell population.

In some embodiments the methods described herein include administering about 2×10⁶ cells/cm³ of affected cortex to about 2×10⁷ cells/cm³ of affected cortex. In other embodiments the methods include administering 0.1×10⁶ cells/kg body weight to 5×10⁶ cells/kg body weight.

In some embodiments the human cell population to be administered was culture expanded prior to the administration.

In some embodiments the human cell population was derived from bone marrow, dental pulp, adipose, or pluripotent stem cells. In some embodiments the human cell population was not derived from dental pulp or adipose. In some embodiments the human cell population is a genetically modified human cell population.

In some embodiments the systemic administration of the cell population is intra-arterial administration or intravenous administration.

In some embodiments the methods described herein include administering a thrombolytic agent. In some embodiments the methods described herein avoid administration of a thrombolytic agent. In other embodiments the subject is not administered a thrombolytic agent before or after administration of the human cell population. In other embodiments the methods include administering mannitol. In some embodiments the methods include co-administering mannitol and temozolomide as a single formulation or separately. In other embodiments the methods include administering an anti-inflammatory agent.

In some embodiments the human cell population to be administered is administered a plurality of times. In some embodiments the human cell population is administered once every four or more weeks.

In other embodiments the human cell population is administered a single time.

In some embodiments at least a portion of the cells in the human cell population is labelled for in vivo detection. In some embodiments, where labelled cells are administered to the subject, the method also includes tracking the location of the labelled cells in the subject following the administration.

In some embodiments of any of the above-mentioned methods, the method further includes determining changes in infarct volume and/or activity within the infarct volume following the administration.

The methods described herein are to be taken to apply mutatis mutandis to methods for reducing the risk of a further cerebral infarction.

### BRIEF DESCRIPTION OF THE DRAWINGS

FIG. 1. A line graph representing forelimb placement motor behavioral scores in groups of rats at various time points following medial carotid artery occlusion (MCAO), a model of infarct. The various groups were administered 1×10⁶ human MPCs intravenously at the indicated time points following MCAO treatment. Note: lower numbers signify improved motor behavior. Administration of MPCs at 6 hours (p<0.01), 12 hours (p<0.01), 24 hours (p<0.001), 48 hours (p<0.01), and 7 days (p<0.01) post MCAO significantly improved forelimb recovery compared to vehicle administration.

FIG. 2. A line graph representing hindlimb placement motor behavioral scores in groups of rats at various time points following MCAO. The various groups were administered 1×10⁶ human MPCs intravenously at the indicated time points following MCAO treatment. Administration of MPCs at 6 hours (p<0.001), 12 hours (p<0.01), 24 hours (p<0.001), and 48 hours (p<0.001) post MCAO significantly improved hindlimb recovery compared to vehicle administration.

FIG. 3. A line graph representing body swing motor behavioral scores in groups of rats at various time points following MCAO. The various groups were administered 1×10⁶ human MPCs intravenously at the indicated time points following MCAO treatment. Administration of huMPCs at 6 hours (p<0.05), 12 hr (p<0.05), 48 hours (p<0.01), and 7 days (p<0.01) post MCAO significantly improved body swing recovery compared to vehicle administration.

FIG. 4. A line graph representing body weight after MCAO. There were no significant differences in body weight comparing the MPC treated groups to the vehicle group.

FIG. 5. A schematic summary of an MRI imaging study of cortical responsiveness to tactile stimuli in rats following MCAO.

FIG. 6. A schematic summary of MRI imaging setup for the MCAO rat study.

FIG. 7. A bar graph showing measurements (mean±SEM) at time of MRI on day 8 for the infarct volume (top panel), and the infarct volume as a percent of whole brain (bottom panel). The MPC treated group had statistically smaller infarct volume compared to vehicle treated group (p<0.05). Additionally, the infarct volume as a percent of whole brain was significantly smaller in the MPC treated group (p<0.05).

FIG. 8. A bar graph showing activation in primary and secondary motor cortex due to left (contralateral) forepaw stimulus in vehicle and MPC-treated groups (bottom panel) and primary and secondary somatosensory cortex (bottom panel). A significantly greater level of activation in primary motor cortex was observed in the MPC-treated group than the vehicle-treated group (p<0.05).

FIG. 9. A bar graph showing the level of cortical activation within the infarct cortical volume in response to contralateral tactile stimulation in vehicle-treated and MPC-treated groups. The level of cortical activation within the infarct was significantly greater in the MPC-treated group than in the vehicle-treated group (p<0.01).

### DETAILED DESCRIPTION

### General Techniques and Selected Definitions

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example of the disclosure is to be applied mutatis mutandis to each and every other example unless specifically stated otherwise.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure.

The present disclosure is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson etal, pp35-81; Sproat etal, pp 83-115; and Wu etal, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J.F. Ramalho Ortigao, “The Chemistry of Peptide Synthesis” In: Knowledge database of Access to Virtual Laboratory web site (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wunsch, E., ed. (1974) Synthese von Peptiden in Houben-Weyls Metoden der Organischen Chemie (Miller, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

As used herein, the term “cerebral infarction” shall be taken to mean loss of brain function(s), usually rapidly developing, that is due to a disturbance in blood flow to the brain or brainstem. The disturbance can be ischemia (lack of blood) caused by, e.g., thrombosis or embolism (referred to herein as an “ischemic cerebral infarction,” or can be due to a hemorrhage (referred to herein as a “hemorrhagic cerebral infarction). In one example, the loss of brain function is accompanied by neuronal cell death. In one example, the cerebral infarction is caused by a disturbance or loss of blood from to the cerebrum or a region thereof. In one example, a cerebral infarction is a neurological deficit of cerebrovascular cause that persists beyond 24 hours or is interrupted by death within 24 hours (as defined by the World Health Organization). Persistence of symptoms beyond 24 hours separates cerebral infarction from Transient Ischemic Attack (TIA), in which symptoms persist for less than 24 hours. Symptoms of cerebral infarction include hemiplegia (paralysis of one side of the body); hemiparesis (weakness on one side of the body); muscle weakness of the face; numbness; reduction in sensation; altered sense of smell, sense of taste, hearing, or vision; loss of smell, taste, hearing, or vision; drooping of an eyelid (ptosis); detectable weakness of an ocular muscle; decreased gag reflex; decreased ability to swallow; decreased pupil reactivity to light; decreased sensation of the face; decreased balance; nystagmus; altered breathing rate; altered heart rate; weakness in sternocleidomastoid muscle with decreased ability or inability to turn the head to one side; weakness in the tongue; aphasia (inability to speak or understand language); apraxia (altered voluntary movements); a visual field defect; a memory deficit; hemineglect or hemispatial neglect (deficit in attention to the space on the side of the visual field opposite the lesion); disorganized thinking; confusion; development of hypersexual gestures; anosognosia (persistent denial of the existence of a deficit); difficulty walking; altered movement coordination; vertigo; disequilibrium; loss of consciousness; headache; and/or vomiting.

The skilled person will be aware that the “cerebrum” includes the cerebral cortex (or cortices of the cerebral hemispheres), the basal ganglia (or basal nuclei) and limbic system.

The term “infarct,” as used herein, refers to a region or volume of brain directly compromised by the process of a cerebral infarction.

The term “cerebral function” includes:
reasoning, planning, parts of speech, movement, emotions, and problem solving (associated with the frontal lobe);
movement, orientation, recognition, perception of stimuli (associated with the parietal lobe);
visual processing (associated with the occipital lobe); and
perception and recognition of auditory stimuli, memory, and speech (associated with the temporal lobe).

As used herein, the term “effective amount” or “therapeutically effective amount” shall be taken to mean a sufficient quantity of the population enriched for the STRO-1⁺ MPCs, and/or progeny cells thereof (equivalently referred to as “culture-expanded STRO-1⁺ MPCs) to alleviate one or more effects of a cerebral infarct, e.g., reduced motor function. A single dose of an “effective amount” does not necessarily have to be sufficient to provide a therapeutic benefit on its own, for example, a plurality of administrations of an effective amount of the population may provide an improved therapeutic benefit.

As used herein, the term “low dose” shall be understood to mean an amount of STRO-1⁺ cells and/or progeny thereof less than 1×10⁶, yet still sufficient to be an “effective amount” as defined herein and/or a “therapeutically effective amount” as defined herein. For example, a low dose comprises 0.5×10⁶ or fewer cells, or 0.4×10⁶ or fewer cells or 0.3 ×10⁶ or fewer cells or 0.1×10⁶ or fewer cells.

As used herein, the term “treat” or “treatment” or “treating” shall be understood to mean administering an amount of cells (systemic) and increasing stimulus-induced cortical activity (e.g., primary cortical activity) in response to sensory input relative to corresponding activity in an untreated subject.

As used herein, the term “normal or healthy individual” shall be taken to mean a subject who has not suffered a cerebral infarction.

As used herein, the term “STRO-1⁺ cells,” as used herein, is equivalent to STRO-1⁺ mesenchymal precursor cells (MPCs) or STRO-1⁺ multipotential cells.

As used herein, the term “progeny thereof” in reference to STRO-1⁺ cells, STRO-1⁺ MPCs, or STRO-1⁺ multipotential cells refers to any of the foregoing cells following their expansion in culture, where such culture expanded (progeny) cells retain the multipotential and therapeutic properties of the starting “primary” STRO-1⁺ cells.

In this specification, the term “effect of cerebral infarction” will be understood to include and provide literal support for one or more of increasing cortical activity (e.g., primary motor cortex activity), increasing cortical activity within the volume of an infarct, and/or reducing infarct volume.

### Mesenchymal Lineage Precursor or Stem Cells (MLPSCs)

In some embodiments a human cell population enriched for MLPSCs to be administered in a method described herein is derived from bone marrow, dental pulp, adipose, or pluripotent stem cells. In some embodiments the human cell populations is not derived from dental pulp or adipose. In some embodiments the human cell population is not derived from dental pulp. In some embodiments the human cell population enriched for MLPSCs is enriched for STRO-1⁺ cells. STRO-1⁺ cells can be found in bone marrow, blood, dental pulp cells, adipose tissue, skin, spleen, pancreas, brain, kidney, liver, heart, retina, brain, hair follicles, intestine, lung, lymph node, thymus, bone, ligament, tendon, skeletal muscle, dermis, and periosteum; and are capable of differentiating into germ lines such as mesoderm and/or endoderm and/or ectoderm. Exemplary sources of STRO-1⁺ cells are derived from bone marrow and/or dental pulp.

In one example, the STRO-1⁺ cells are multipotential cells which are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues. STRO-1⁺ multipotential cells are thus non-hematopoietic progenitor cells which divide to yield daughter multipotential stem cells.

In one example, the STRO-1⁺ cells are enriched from a sample obtained from a human subject, e.g., a subject to be treated or a related subject or an unrelated subject. The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for STRO-1⁺ cells comprises at least about 0.1% or 0.5% or 1% or 2% or 5% or 10% or 15% or 20% or 25% or 30% or 50% or 75% STRO-1⁺ cells. In this regard, the term “population of cells enriched for STRO-1⁺ cells” will be taken to provide explicit support for the term “population of cells comprising X% STRO-1⁺ cells”, wherein X% is a percentage as recited herein. The STRO-1⁺ cells can, in some examples, form clonogenic colonies, e.g. CFU-F (fibroblasts) or a subset thereof (e.g., 50% or 60% or 70% or 70% or 90% or 95%) can have this activity.

In one example, the population of cells is enriched from a cell preparation comprising STRO-1⁺ cells in a selectable form. In this regard, the term “selectable form” will be understood to mean that the cells express a marker (e.g., a cell surface marker) permitting selection of the STRO-1⁺ cells. The marker can be STRO-1, but need not be. For example, as described and/or exemplified herein, cells (e.g., MPCs) expressing STRO-2 and/or STRO-3 (TNAP) and/or STRO-4 and/or VCAM-1 and/or CD146 also express STRO-1 (and can be STRO-1^{bright})Accordingly, an indication that cells are STRO-1⁺ does not mean that the cells are selected by STRO-1 expression. In one example, the cells are selected based on at least STRO-3 expression, e.g., they are STRO-3⁺ (TNAP⁺).

Reference to selection of a cell or population thereof does not require selection from a specific tissue source. As described herein STRO-1⁺ cells can be selected from or isolated from or enriched from a large variety of sources. That said, in some examples, these terms provide support for selection from any tissue comprising STRO-1⁺ cells (e.g., MPCs) or vascularized tissue or tissue comprising pericytes (e.g., STRO-1⁺ pericytes) or any one or more of the tissues recited herein.

In one example, the cells express one or more markers individually or collectively selected from the group consisting of TNAP⁺, VCAM-1⁺, THY-1⁺, CD146⁺, or any combination thereof.

In one example, the cells express or the population of cells is enriched for mesenchymal precursor cells expressing STRO-1⁺ (or STRO-1^{bright}) and CD146⁺.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of peptides, and that, notwithstanding that such numbers or combinations of markers or groups of markers may not be specifically listed herein the accompanying claims may define such combinations or sub-combinations separately and divisibly from any other combination of markers or groups of markers.

In one example, the STRO-1⁺ cells are STRO-1^{bright} (syn. STRO-1^{bri}). In one example, the Stro-1^{bri} cells are preferentially enriched relative to STRO-1^{dim} or STRO-^{intermediate} cells.

For example, the STRO-1^{bright} cells are additionally one or more of TNAP⁺, VCAM-1⁺, THY-1⁺, and/or CD146⁺. For example, the cells are selected for one or more of the foregoing markers and/or shown to express one or more of the foregoing markers. In this regard, a cell shown to express a marker need not be specifically tested, rather previously enriched or isolated cells can be tested and subsequently used, isolated or enriched cells can be reasonably assumed to also express the same marker.

In one example, the mesenchymal precursor cells are perivascular mesenchymal precursor cells as defined in WO 2004/85630.

A cell that is referred to as being “positive” for a given marker it may express either a low (lo or dim) or a high (bright, bri) level of that marker depending on the degree to which the marker is present on the cell surface, where the terms relate to intensity of fluorescence or other marker used in the sorting process of the cells. The distinction of lo (or dim or dull) and bri will be understood in the context of the marker used on a particular cell population being sorted. A cell that is referred to as being “negative” for a given marker is not necessarily completely absent from that cell. This term means that the marker is expressed at a relatively very low level by that cell, and that it generates a very low signal when detectably labeled or is undetectable above background levels, e.g., levels detected suing an isotype control antibody.

The term “bright”, when used herein, refers to a marker on a cell surface that generates a relatively high signal when detectably labeled. Whilst not wishing to be limited by theory, it is proposed that “bright” cells express more of the target marker protein (for example the antigen recognized by STRO-1) than other cells in the sample. For instance, STRO-1^{bri} cells produce a greater fluorescent signal, when labeled with a FITC-conjugated STRO-1 antibody as determined by fluorescence activated cell sorting (FACS) analysis, than non-bright cells (STRO-1^{dull/dim}). For example, “bright” cells constitute at least about the 0.1% most brightly labeled cells within a distribution of labeled cell intensities. In other examples, “bright” cells constitute at least about the 0.1%, at least about 0.5%, at least about 1%, at least about 1.5%, or at least about 2%, most brightly STRO-1 labelled cells in the starting sample. In an example, STRO-1^{bright} cells have 2 log magnitude higher expression of STRO-1 surface expression relative to “background”, namely cells that are STRO-1⁻. By comparison, STRO-1^{dim} and/or STRO-1^{intermediate} cells have less than 2 log magnitude higher expression of STRO-1 surface expression, typically about 1 log or less than “background”.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In an example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in an example of the disclosure, the STRO-1⁺ cells are capable of giving rise to clonogenic CFU-F.

In one example, a significant proportion of MLPSCs, e.g., STRO-1⁺ multipotential cells are capable of differentiation into at least two different germ lines. Non-limiting examples of the lineages to which the multipotential cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In another example, the MLPSCs, e.g., STRO-1⁺ cells, are not capable of giving rise, upon culturing, to hematopoietic cells.

In one example, the cells are taken from the subject to be treated, culture-expanded in vitro using standard techniques and used to obtain expanded cells for administration to the subject as an autologous or allogeneic composition. In an alternative example, cells of one or more of the established human cell lines are used. In some embodiments the MLPSCs, e.g., cells are obtained by differentiation of pluripotent stem cells, e.g., human induced pluripotent stem cells (hiPSCs) See, e.g., Dayem et al (2019), International Journal of Molecular Science, 20(8):E1922.

In some embodiments, progeny (expanded) cells are obtained after about 2, about 3, about 4, about 5, about 6, about 7, about 8, about 9, or about 10 passages from the parental population. However, the progeny cells may be obtained after any number of passages from the parental population.

The progeny cells may be obtained by culturing in any suitable medium. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. The term “medium” also refers to material that is intended for use in a cell culture, even if it has not yet been contacted with cells. In other words, a nutrient rich liquid prepared for bacterial culture is a medium. A powder mixture that when mixed with water or other liquid becomes suitable for cell culture may be termed a “powdered medium”.

In an example, progeny cells useful for the methods of the disclosure are obtained by isolating or enriching TNAP⁺ STRO-1⁺ cells from bone marrow using magnetic beads labeled with the STRO-3 antibody, and then culture expanding the isolated cells (see Gronthos et al. Blood 85: 929-940, 1995 for an example of suitable culturing conditions).

In some embodiments, expanded MLPSCs may express one or more markers collectively or individually selected from the group consisting of LFA-3, THY-1, VCAM-1, ICAM-1, PECAM-1, P-selectin, L-selectin, 3G5, CD49a/CD49b/CD29, CD49c/CD29, CD49d/CD29, CD 90, CD29, CD18, CD61, integrin beta 6-19, thrombomodulin, CD10, CD13, SCF, PDGF-R, EGF-R, IGF1-R, NGF-R, FGF-R, Leptin-R (STRO-2=Leptin-R), RANKL, STRO-4 (HSP-90β), STRO-1^{bright} and CD146 or any combination of these markers.

Methods for preparing enriched populations of some MLPSCs, e.g., STRO-1⁺ multipotential cells and their culture expansion are described in WO 01/04268 and WO 2004/085630. In an in vitro context STRO-1⁺ multipotential cells will rarely be present as an absolutely pure preparation and will generally be present with other cells that are tissue specific committed cells (TSCCs). WO 01/04268 refers to harvesting such cells from bone marrow at purity levels of about 0.1% to 90%. The population comprising MPCs from which progeny are derived may be directly harvested from a tissue source, or alternatively it may be a population that has already been expanded ex vivo.

For example, the progeny may be obtained from a harvested, unexpanded, population of substantially purified STRO-1⁺ multipotential cells, comprising at least about 0.1, 1, 5, 10, 20, 30, 40, 50, 60, 70, 80 or 95% of total cells of the population in which they are present. This level may be achieved, for example, by selecting for cells that are positive for at least one marker individually or collectively selected from the group consisting of TNAP, STRO-4 (HSP-90β), STRO-1^{bright}, 3G5⁺, VCAM-1, THY-1, CD146 and STRO-2.

A STRO-1⁺ cell starting population may be derived from any one or more tissue types set out in WO 01/04268 or WO 2004/085630, namely bone marrow, dental pulp cells, adipose tissue and skin, or perhaps more broadly from adipose tissue, teeth, dental pulp, skin, liver, kidney, heart, retina, brain, hair follicles, intestine, lung, spleen, lymph node, thymus, pancreas, bone, ligament, bone marrow, tendon and skeletal muscle. In some preferred embodiments, a population enriched for STRO-1⁺ cells is derived from bone marrow, dental pulp, adipose, or pluripotent stem cells.

It will be understood that in performing methods described in the present disclosure, separation of cells carrying any given cell surface marker can be effected by a number of different methods, however, exemplary methods rely upon binding a binding agent (e.g., an antibody or antigen binding fragment thereof) to the marker concerned followed by a separation of those that exhibit binding, being either high level binding, or low level binding or no binding. The most convenient binding agents are antibodies or antibody-based molecules, for example monoclonal antibodies or based on monoclonal antibodies (e.g., proteins comprising antigen binding fragments thereof) because of the specificity of these latter agents. Antibodies can be used for both steps, however other agents might also be used, thus ligands for these markers may also be employed to enrich for cells carrying them, or lacking them.

The antibodies or ligands may be attached to a solid support to allow for a crude separation. In one example, the separation techniques maximize the retention of viability of the fraction to be collected. Various techniques of different efficacy may be employed to obtain relatively crude separations. The particular technique employed will depend upon efficiency of separation, associated cytotoxicity, ease and speed of performance, and necessity for sophisticated equipment and/or technical skill. Procedures for separation may include, but are not limited to, magnetic separation, using antibody-coated magnetic beads, affinity chromatography and “panning” with antibody attached to a solid matrix. Techniques providing accurate separation include but are not limited to FACS. Methods for performing FACS will be apparent to the skilled artisan.

Antibodies against each of the markers described herein are commercially available (e.g., monoclonal antibodies against STRO-1 are commercially available from R&D Systems, USA), available from ATCC or other depositary organization and/or can be produced using art recognized techniques.

In one example, a method for isolating STRO-1⁺ cells comprises a first step being a solid phase sorting step utilizing for example magnetic activated cell sorting (MACS) recognizing high level expression of STRO-1. A second sorting step can then follow, should that be desired, to result in a higher level of precursor cell expression as described in patent specification WO 01/14268. This second sorting step might involve the use of two or more markers.

In some embodiments the MLSCs are mesenchymal stem cells (MSCs). The MSCs may be a homogeneous composition or may be a mixed cell population enriched in MSCs. Homogeneous MSC compositions may be obtained by culturing adherent bone marrow or periosteal cells, and the MSCs may be identified by specific cell surface markers which are identified with unique monoclonal antibodies. A method for obtaining a cell population enriched in MSCs using plastic adherence technology is described, for example, in U.S. Pat. No. 5,486,359. MSC prepared by conventional plastic adherence isolation relies on the non-specific plastic adherent properties of CFU-F. Alternative sources for MSCs include, but are not limited to, blood, skin, cord blood, muscle, fat, bone, and perichondrium.

The mesenchymal lineage precursor or stem cells may be cryopreserved prior to administration to a subject.

A method for obtaining MLPSCs, e.g., mesenchymal stem cells, might also include the harvesting of a source of the cells before the first enrichment step using known techniques. Thus the tissue will be surgically removed. Cells comprising the source tissue will then be separated into a so called single cells suspension. This separation may be achieved by physical and or enzymatic means.

Once a suitable MLPSC population has been obtained, it may be cultured or expanded by any suitable means.

In some embodiments, the cells are taken from the subject to be treated, cultured in vitro using standard techniques and used to obtain expanded cells for administration to the subject as an autologous or a different subject as an allogeneic composition.

Cells useful for the methods of the present disclosure may be stored before use. Methods and protocols for preserving and storing of eukaryotic cells, and in particular mammalian cells, are known in the art (cf., for example, Pollard, J. W. and Walker, J. M. (1997) Basic Cell Culture Protocols, Second Edition, Humana Press, Totowa, N.J.; Freshney, R. I. (2000) Culture of Animal Cells, Fourth Edition, Wiley-Liss, Hoboken, N.J.). Any method maintaining the biological activity of the isolated stem cells such as mesenchymal stem/progenitor cells, or progeny thereof, may be utilized in connection with the present disclosure. In one example, the cells are maintained and stored by using cryo-preservation.

### Modified Cells

In one example, the MLPSCs and/or progeny cells thereof are genetically modified, e.g., to express and/or secrete a protein of interest. For example, the cells are engineered to express a protein useful in the treatment of movement disorders or other effects of cerebral infarction, such as, vascular endothelial growth factor (VEGF), erythropoietin, brain-derived growth factor (BDNF), or insulin-like growth factor (IGF-1), as reviewed in, e.g., Larpthaveesarp et al (2015), Brain Science 5(2):165-177.

Methods for genetically modifying a cell will be apparent to the skilled artisan. For example, a nucleic acid that is to be expressed in a cell is operably-linked to a promoter for inducing expression in the cell. For example, the nucleic acid is linked to a promoter operable in a variety of cells of a subject, such as, for example, a viral promoter, e.g., a CMV promoter (e.g., a CMV-IE promoter) or a SV-40 promoter. Additional suitable promoters are known in the art and shall be taken to apply mutatis mutandis to the present example of the disclosure.

In one example, the nucleic acid is provided in the form of an expression construct. As used herein, the term “expression construct” refers to a nucleic acid that has the ability to confer expression on a nucleic acid (e.g. a reporter gene and/or a counter-selectable reporter gene) to which it is operably connected, in a cell. Within the context of the present disclosure, it is to be understood that an expression construct may comprise or be a plasmid, bacteriophage, phagemid, cosmid, virus sub-genomic or genomic fragment, or other nucleic acid capable of maintaining and/or replicating heterologous DNA in an expressible format.

Methods for the construction of a suitable expression construct for performance of the disclosure will be apparent to the skilled artisan and are described, for example, in Ausubel et al (In: Current Protocols in Molecular Biology. Wiley Interscience, ISBN 047 150338, 1987) or Sambrook et al (In: Molecular Cloning: Molecular Cloning: A Laboratory Manual, Cold Spring Harbor Laboratories, New York, Third Edition 2001). For example, each of the components of the expression construct is amplified from a suitable template nucleic acid using, for example, PCR and subsequently cloned into a suitable expression construct, such as for example, a plasmid or a phagemid.

Vectors suitable for such an expression construct are known in the art and/or described herein. For example, an expression vector suitable for use in a method of the present disclosure in a mammalian cell is, for example, a vector of the pcDNA vector suite supplied by Invitrogen, a vector of the pCI vector suite (Promega), a vector of the pCMV vector suite (Clontech), a pM vector (Clontech), a pSI vector (Promega), a VP 16 vector (Clontech) or a vector of the pcDNA vector suite (Invitrogen).

The skilled artisan will be aware of additional vectors and sources of such vectors, such as, for example, Life Technologies Corporation, Clontech or Promega.

Means for introducing the isolated nucleic acid molecule or a gene construct comprising same into a cell for expression are known to those skilled in the art. The technique used for a given organism depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

Alternatively, an expression construct of the disclosure is a viral vector. Suitable viral vectors are known in the art and commercially available. Conventional viral-based systems for the delivery of a nucleic acid and integration of that nucleic acid into a host cell genome include, for example, a retroviral vector, a lentiviral vector or an adeno-associated viral vector. Alternatively, an adenoviral vector is useful for introducing a nucleic acid that remains episomal into a host cell. Viral vectors are an efficient and versatile method of gene transfer in target cells and tissues. Additionally, high transduction efficiencies have been observed in many different cell types and target tissues.

For example, a retroviral vector generally comprises cis-acting long terminal repeats (LTRs) with packaging capacity for up to 6-10 kb of foreign sequence. The minimum cis-acting LTRs are sufficient for replication and packaging of a vector, which is then used to integrate the expression construct into the target cell to provide long term expression. Widely used retroviral vectors include those based upon murine leukemia virus (MuLV), gibbon ape leukemia virus (GaLV), simian immunodeficiency virus (SrV), human immunodeficiency virus (HIV), and combinations thereof (see, e.g., Buchscher et al., J Virol. 56:2731-2739 (1992); Johann et al, J. Virol. 65:1635-1640 (1992); Sommerfelt et al, Virol. 76:58-59 (1990); Wilson et al, J. Virol. 63:274-2318 (1989); Miller et al., J. Virol. 65:2220-2224 (1991); PCT/US94/05700; Miller and Rosman BioTechniques 7:980-990, 1989; Miller, A. D. Human Gene Therapy 7:5-14, 1990; Scarpa et al Virology 75:849-852, 1991; Burns et al. Proc. Natl. Acad. Sci USA 90:8033-8037, 1993).

Various adeno-associated virus (AAV) vector systems have also been developed for nucleic acid delivery. AAV vectors can be readily constructed using techniques known in the art. See, e.g., U.S. Pat. Nos. 5,173,414 and 5,139,941; International Publication Nos. WO 92/01070 and WO 93/03769; Lebkowski et al. Molec. Cell. Biol. 5:3988-3996, 1988; Vincent et al. (1990) Vaccines 90 (Cold Spring Harbor Laboratory Press); Carter Current Opinion in Biotechnology 5:533-539, 1992; Muzyczka. Current Topics in Microbiol, and Immunol. 158:97-129, 1992; Kotin, Human Gene Therapy 5:793-801, 1994; Shelling and Smith Gene Therapy 7:165-169, 1994; and Zhou et al. J Exp. Med. 179:1867-1875, 1994.

Additional viral vectors useful for delivering an expression construct of the disclosure include, for example, those derived from the pox family of viruses, such as vaccinia virus and avian poxvirus or an alphavirus or a conjugate virus vector (e.g. that described in Fisher-Hoch et al., Proc. Natl Acad. Sci. USA 56:317-321, 1989).

In some embodiments at least a portion of the cells to be administered are labelled to facilitate non-invasive detection, localization, and/or tracking of the administered labelled cells following their administration. In some embodiments the cells are genetically modified to express a reporter protein that can be detected non-invasively in vivo, e.g., monomeric far red fluorescent proteins. See, e.g., Wannier et al. (2018), PNAS, 115 (48) E11294-E11301. In other embodiments the cells to be administered are labelled by non-genetic means, e.g., using a vital tracking label that can be introduced into at least a portion of the cells to be administered, and subsequently detected non-invasively in vivo. An example of a suitable tracking label is Molday ION™ Rhodamine B (MIRB) (available from Biophysics Assay Laboratory, Inc.), an iron oxide-based superparamagnetic Mill contrast reagent having a colloidal size of 35 nm designed for cell labeling and MRI tracking and does not require transfection reagents for efficient cell labeling. Tracking can be visualized by Mill or fluorescence.

### Models of Cerebral Infarct

There are various known techniques for inducing an ischemic cerebral infarction in a non-human animal subject, such as, aorta/vena cava occlusion, external neck torniquet or cuff, hemorrhage or hypotension, intracranial hypertension or common carotid artery occlusion, two-vessel occlusion and hypotension, four-vessel occlusion, unilateral common carotid artery occlusion (in some species only), endothelin-1-induced constriction of arteries and veins, middle cerebral artery occlusion, spontaneous brain infarction (in spontaneously hypertensive rats), macrosphere embolization, blood clot embolization or microsphere embolization. Hemorrhagic cerebral infarction can be modeled by infusion of collagenase into the brain.

In one example, the model of cerebral infarction comprises middle cerebral artery occlusion to produce an ischemic cerebral infarction.

To test the ability of a population and/or progeny to treat the effects of cerebral infarction, the population and/or progeny are administered following induction of cerebral infarction, e.g., within 1 hour to 1 day of cerebral infarction. Following administration an assessment of cerebral function and/or movement disorder is made, e.g., on several occasions.

Methods of assessing cerebral function and/or movement disorders will be apparent to the skilled artisan and include, for example, rotarod, elevated plus maze, open-field, Morris water maze, T-maze, the radial arm maze, assessing movement (e.g., area covered in a period of time), tail flick or De Ryck's behavioral test (De Ryck et al., Cerebral infarction. 20:1383-1390, 1989). Additional tests will be apparent to the skilled artisan and/or described herein. Likewise, models of HIE are known in the art. See, e.g., Millar et al (2017), Frontiers in Cellular Neuroscience, 11(78): 1-36.

In another example, the effect of administered cells on sensory stimulus-evoked cortical activity, infarct volume, or cortical activity within infarct by imaging techniques. In some preferred embodiments, magnetic resonance imaging (MM), and particularly functional MM (fMRI) techniques such as Blood Oxygen Level-Dependent (BOLD) imaging are useful for making such assessments. PET and CT can also be used to make such assessments.

Motor behavior assay functional assessments alone or in combination with imaging techniques may be used to assess the therapeutic effects of the cellular compositions described herein. Such behavioral assays include, but are not limited to, limb placement, rotorod, grid walking, and elevated body swing. See, e.g., Schaar et al (2010), Experimental & Translational Stroke Medicine, 2:13; and Borlongan et al (1995), Physiology & Behavior, 58(5):909-917.

### Cellular Compositions

In one example of the present disclosure MLPSCs, e.g., STRO-1⁺ cells and/or progeny cells thereof are administered in the form of a composition. In one example, such a composition comprises a pharmaceutically acceptable carrier and/or excipient.

The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. In an example, such a media composition does not induce any adverse effects in a subject to whom it is administered. Further examples include cryopreservative media, e.g., physiological media comprising one or more cryoprotective agents such as cryoprotective polyols such as dimethylsulfoxide (DMSO), trehalose, or combinations thereof.

Exemplary carriers and excipients do not adversely affect the viability of a cell and/or the ability of a cell to reduce, prevent or delay an effect of cerebral infarction.

In one example, the carrier or excipient provides a buffering activity to maintain the cells at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

The cellular compositions useful for methods described herein may be administered alone or as admixtures with other cells. Cells that may be administered in conjunction with the compositions of the present disclosure include, but are not limited to, other multipotent or pluripotent cells or stem cells, or bone marrow cells. The cells of different types may be admixed with a composition of the disclosure immediately or shortly prior to administration, or they may be co-cultured together for a period of time prior to administration.

In one example, the composition comprises an effective amount or a therapeutically or prophylactically effective amount of cells. For example, the composition comprises about 1×10⁵ MLPSCs/kg to about 1×10⁷ MLPSCs/kg or about 1×10⁶ MLPSCs/kg to about 5×10⁶ MLPSCs/kg. In another example, the composition comprises about 1×10⁵ STRO-1⁺ cells/kg to about 1×10⁷ STRO-1⁺ cells/kg or about 1×10⁶ STRO-1⁺ cells/kg to about 5×10⁶ STRO-1⁺ cells/kg. The exact amount of cells to be administered is dependent upon a variety of factors, including the age, weight, and sex of the patient, and the extent and severity of the cerebral infarction and/or site of the cerebral infarction.

In some embodiments, a low dose of cells is administered systemically to the subject. Exemplary dosages include between about 0.1×10⁶ and 2×10⁶ MLPSCs per kg, for example, between about 0.5×10⁵ and 2×10⁶ MLPSCs per kg, such as, between about 0.7×10⁵ and 1.5×10⁶ MLPSCs per kg, for example, about 0.8×10⁵, 1.0×10⁶, 1.2×10⁶, or 1.4×10⁶ MLPSCs/kg.

In other embodiments systemic dosing is based on an assessed volume of the infarct, e.g., about 2×10⁶ MLPSCs/cm³ of affected cortex to about 2×10⁷ MLPSCs/cm³ of affected cortex, e.g., 3×10⁶, 4×10⁶, 5×10⁶, 8×10⁶, 1.2×10⁷, 1.5×10⁷, or another number of cells/cm³ from about 2 ×10⁶ MLPSCs/cm³ to about 2×10⁷ MLPSCs/cm³.

In some examples of the disclosure, it may not be necessary or desirable to immunosuppress a patient prior to initiation of therapy with cellular compositions. Accordingly, infusion with allogeneic, MLPSCs, e.g., STRO-1⁺ cells or progeny thereof may be tolerated in some instances.

However, in other instances it may be desirable or appropriate to pharmacologically immunosuppress a patient prior to initiating cell therapy and/or reduce an immune response of a subject against the cellular composition. This may be accomplished through the use of systemic or local immunosuppressive agents. As an alternative, the cells may be genetically modified to reduce their immunogenicity.

### Additional Components of Compositions

The MLPSCs or progeny thereof may be administered with other beneficial drugs or biological molecules (growth factors, trophic factors). When administered with other agents, they may be administered together in a single pharmaceutical composition, or in separate pharmaceutical compositions, simultaneously or sequentially with the other agents (either before or after administration of the other agents). Bioactive factors which may be co-administered include anti-apoptotic agents (e.g., EPO, EPO mimetibody, TPO, IGF-I and IGF-II, HGF, caspase inhibitors); anti-inflammatory agents (e.g., p38 MAPK inhibitors, TGF-beta inhibitors, statins, IL-6 and IL-1 inhibitors, PEMIROLAST, TRANILAST, REMICADE, SIROLIMUS, and NSAIDs (non-steroidal anti-inflammatory drugs; e.g., TEPDXALIN, TOLMETIN, SUPROFEN); immunosupressive/immunomodulatory agents (e.g., calcineurin inhibitors, such as cyclosporine, tacrolimus; mTOR inhibitors (e.g., SIROLIMUS, EVEROLIMUS); anti-proliferatives (e.g., azathioprine, mycophenolate mofetil); corticosteroids (e.g., prednisolone, hydrocortisone); antibodies such as monoclonal anti-IL-2Ralpha receptor antibodies (e.g., basiliximab, daclizumab), polyclonal anti-T-cell antibodies (e.g., anti-thymocyte globulin (ATG); anti-lymphocyte globulin (ALG); monoclonal anti-T cell antibody OKT3)); anti-thrombogenic agents (e.g., heparin, heparin derivatives, urokinase, PPack (dextrophenylalanine proline arginine chloromethylketone), antithrombin compounds, platelet receptor antagonists, anti-thrombin antibodies, anti-platelet receptor antibodies, aspirin, dipyridamole, protamine, hirudin, prostaglandin inhibitors, and platelet inhibitors); and anti-oxidants (e.g., probucol, vitamin A, ascorbic acid, tocopherol, coenzyme Q-10, glutathione, L-cysteine, N-acetylcysteine) as well as local anesthetics. In some embodiments a cellular composition to be administered includes an anti-inflammatory agent. In other embodiments a cellular composition to be administered includes a thrombolytic agent.

In some embodiments cellular compositions include an agent to transiently disrupt the blood-brain barrier (BBB). In some embodiments the cellular compositions to be administered include mannitol. Alternatively, mannitol is administered shortly before or after administration of the cellular compositions, e.g., within about one hour.

In some embodiments, a composition as described herein according to any example comprises a factor for improving cerebral function and/or regenerating cerebral neurons and/or treating motor dysfunction, e.g., a trophic factor.

Alternatively, or in addition, cells, and/or a composition as described herein according to any example is combined with a known treatment of cerebral infarction effects, e.g., physiotherapy and/or speech therapy.

In one example, a pharmaceutical composition as described herein according to any example comprises a compound used to treat effects of a cerebral infarction. Alternatively, a method of treatment/prophylaxis as described herein according to any example of the disclosure additionally comprises administering a compound used to treat effects of a cerebral infarction. Exemplary compounds are described herein and are to be taken to apply mutatis mutandis to these examples of the present disclosure.

In another example, a composition as described herein according to any example additionally comprises a factor that induces or enhances differentiation of a progenitor cell into a vascular cell. Exemplary factors include, vascular endothelial growth factor (VEGF), platelet derived growth factor (PDGF; e.g., PDGF-BB), and FGF.

### Medical Devices

The present disclosure also provides medical devices for use or when used in a method as described herein according to any example. For example, the present disclosure provides a syringe or catheter or other suitable delivery device comprising STRO-1⁺ cells and/or progeny cells thereof and/or a composition as described herein according to any example. Optionally, the syringe or catheter is packaged with instructions for use in a method as described herein according to any example.

### Administration

In some embodiments a subject to be treated is suffering from an ischemic cerebral infarction. In particular embodiments the subject to be treated is a neonatal subject suffering from hypoxic ischemic encephalopathy (HIE). In other embodiments the cerebral infarction is a hemorrhagic cerebral infarction. In some preferred embodiments the subject to be treated is a human subject.

In preferred embodiments, MLPSCs, e.g., STRO-1⁺ cells, MSCs, or progeny thereof areadministered systemically.

In preferred embodiments, the MLPSCs are delivered to the blood stream of a subject, e.g., parenterally. Exemplary routes of parenteral administration include, but are not limited to, intraarterial, intravenous, intraperitoneal, or intrathecal. In some preferred embodiments, a population of cells enriched for MLPSCs or progeny thereof is delivered intra-arterially, into an aorta, into an atrium or ventricle of the heart.

In the case of cell delivery to an atrium or ventricle of the heart, cells can be administered to the left atrium or ventricle to avoid complications that may arise from rapid delivery of cells to the lungs.

In one embodiment, the population is administered into the carotid artery.

Selecting an administration regimen for a therapeutic formulation depends on several factors, including the serum or tissue turnover rate of the entity, the level of symptoms, and the immunogenicity of the entity.

In one example, MLPSCs or progeny thereof are delivered as a single bolus dose. Alternatively, STRO-1⁺ cells or progeny thereof are administered by continuous infusion, or by doses at intervals of, e.g., one day, one week, or 1-7 times per week. An exemplary dose protocol is one involving the maximal dose or dose frequency that avoids significant undesirable side effects. A total weekly dose depends on the type and activity of the factors/cells being used. Determination of the appropriate dose is made by a clinician, e.g., using parameters or factors known or suspected in the art to affect treatment or predicted to affect treatment. Generally, the dose begins with an amount somewhat less than the optimum dose and is increased by small increments thereafter until the desired or optimum effect is achieved relative to any negative side effects.

In some embodiments a cellular composition described herein (e.g., a human cell population enriched for MLPSCs, e.g., STRO-1⁺ MPCs, STRO-1^{bright} MPCs), or MSCs is administered systemically at about 24 hours or less following cerebral infarction, e.g., at about 1 hour, 2 hours, 3 hours, 4 hours, 5 hours, 6 hours, 7 hours, 8 hours, 9 hours, 12 hours, 16 hours, 18 hours, or another time from about 1 hours to about 24 hours. In other embodiments the cellular composition is administered after 24 hours, e.g., about 25 hours to one month following the infarct, e.g., 26 hours, 28 hours, 48 hours, 72 hours, 96 hours, one week, two weeks, three weeks, or another time point from after 24 hours to about one month following cerebral infarction in the subject to be treated. In some embodiments the cellular composition is administered systemically from after 24 hours to about 48 hours. In other embodiments the cellular composition is administered systemically from about 48 hours to two weeks. In some embodiments, where a cellular composition described herein is to be administered within about 24 hours or less following a cerebral infarction, the subject being treated is not administered a thrombolytic agent either separately (before or after administration of the cells) or as part of the cellular composition itself.

In some embodiments following administration of cells that are suitably labelled for in vivo detection to a subject, as described herein, the distribution of cells in the subject at one or more time points is determined at about six hours to one month following administration of the labelled cells, e.g., at 12 hours, 24 hours, two days, three days, four days, one week, two weeks, three weeks, four weeks, six weeks, seven weeks, or another time point from about six hours to about two months.

In some embodiments, following administration, changes in the volume of the infarct and/or in activity of the infarct in the treated subject at determined over a period from at least 12 hours to six months, e.g., 18 hours, one day, two days, three days, four days, one week, two weeks, three weeks, four weeks, two months, three months, four months, five months, or another period from about 12 hours to about six months. Infarct volume and/or activity within the infarct can be determined with any of number of methods known in the art, e.g., noncontrast head computerized tomography (NCCT) for volume determination and fMRI and analysis of blood oxygen-level-dependent (BOLD) signals within the infarct region.

The present disclosure includes the following non-limiting examples.

### EXAMPLES

### Example 1

### Treatment with Human MPCs Improves Motor Function in a Rodent Model of Infarction

### Animals, Housing and Diet

Eight-four male nude rats (RNU Rats, Taconic, IBU051001C) 250 to 275 g arrived 7-10 days prior to surgery. They were allowed free access to food and water throughout the study. Animals were assigned sequential identification numbers using permanent marker on the tail. The animals were observed the day prior to surgery, and those appearing to be in poor health were excluded from the study.

Animals were housed in rooms provided with filtered air at a temperature of 21±2° C. and 50% ±20% relative humidity. The room was on an automatic timer for a light/dark cycle of 12 hours on and 12 hours off with no twilight. Shepherd's® ¼″ premium corn cob was used for bedding and 1 Nylabone® (3.5″, Dura bones Petite) was put in each cage. Animals were fed with Lab Diet® 5001 chow. Water was provided ad libitum.

The animals were housed two per cage before and after surgery, unless severe aggression or injury was displayed, or death of cage mate, in which case animals were housed singly.

### Study Design

### Animal Preparation

Seventy-two adult male nude rats as described above were used for the study. All rats were housed and handled for behavioral assessment for seven days prior to surgery for acclimation purposes. At the end of the handling period, rats were randomized and assigned to different groups.

### Surgical Preparation

### Middle Cerebral Artery Occlusion (MCAO), Tamura Model

Focal cerebral infarcts were made by permanent occlusion of the proximal right middle cerebral artery (MCA) using a modification of the method of Tamura et al. Male nude rats (250-350 g at the time of surgery) were anesthetized with 2-3% isofluorane in the mixture of N₂O:O₂ (2:1), and were maintained with 1-1.5% isofluorane in the mixture of N₂O:O₂ (2:1). The temporalis muscle was bisected and reflected through an incision made midway between the eye and the eardrum canal. The proximal MCA was exposed through a subtemporal craniectomy without removing the zygomatic arch and without transecting the facial nerve. The artery was then occluded by microbipolar coagulation from just proximal to the olfactory tract to the inferior cerebral vein, and was transected. Body temperature was maintained at 37.0±1° C. throughout the entire procedure. Buprenorphine SR (0.9-1.2 mg/kg, ZooPharm) as analgesia, and Cefazolin (40-50 mg/kg, Hospira) were given at this time before the MCAO surgery.

### Dosing

Cells (hMPC, TAN 2178, Lot #2011CC043) and Vehicle (Cryomedia, Lot #2012CC034) were sent from the Sponsor by dry shipper and stored in liquid nitrogen vapor phase. Cryopreserved hPMCs were thawed just prior to injection as per the following protocols. hMPCs, (1×10⁶ in 0.17 mL) or vehicle (0.17 mL) were administered by tail vein injection at 6 hours, 12 hours, 24 hours, 48 hours, or 7 days following MCAO. The cell suspensions were delivered over approximately 20 seconds. On days when behavioral testing and cell administration were to be given on the same day, cells were always administered after behavioral testing.

### Randomization and Blinding

Animals treated at 24 hours were randomly assigned to receive cells or vehicle using quickcalcs available online at www.graphpad.com/quickcalcs/randomize2.cfm. The other animals were assigned treatment group in a manner to equally distribute treatments into surgical days and maximize the number of animals that could be administered cells from a single vial of thawed cells. The same investigator performed all of the animal surgeries and behavioral assessments, and was blinded to the Study Schedule and treatment assignment of each animal.

### Behavioral Tests

Functional activities were evaluated using limb placing and body swing behavioral tests. These tests were performed one day before MCAO (Day −1), one day (Day 1) and three (Day 3), seven (Day 7), fourteen (Day 14), twenty-one (Day 21) and twenty-eight (Day 28) days after MCAO (Day 0 =day of MCAO1. Limb Placing

Limb placing tests were divided into both forelimb and hindlimb tests. For the forelimb-placing test, the examiner held the rat close to a tabletop and scored the rat's ability to place the forelimb on the tabletop in response to whisker, visual, tactile, or proprioceptive stimulation. Similarly, for the hindlimb placing test, the examiner assessed the rat's ability to place the hindlimb on the tabletop in response to tactile and proprioceptive stimulation. Separate sub-scores were obtained for each mode of sensory input (half-point designations possible), and added to give total scores (for the forelimb placing test: 0=normal, 12=maximally impaired; for the hindlimb placing test: 0=normal; 6=maximally impaired).

### 2. Body Swing Test

The rat was held approximately one inch from the base of its tail. It was then elevated to an inch above a surface of a table. The rat was held in the vertical axis, defined as no more than 10° to either the left or the right side. A swing was recorded whenever the rat moved its head out of the vertical axis to either side. The rat must return to the vertical position for the next swing to be counted. Thirty (30) total swings were counted. A normal rat typically has an equal number of swings to either side. Following focal ischemia, the rat tends to swing to the contralateral (left) side.

### Sacrifice

At twenty-eight days after MCAO, rats were deeply anesthetized with a Ketamine (50-100 mg/kg) and Xylazine (5-10 mg/kg) mixture, intraperitoneally. Rats were then perfused transcardially with normal saline (2 unit/ml heparin) followed by 10% formalin. Brains were removed and stored in 10% formalin. Brains were sent to HistoTechnologies, Inc. and were processed for infarct volume measurement (H&E staining).

### Data Analysis

All data are expressed as mean±S.E.M. Behavioral and body weight data were analyzed by repeated measures of ANOVA (treatment X time). Positive p values for the F-statistic for overall ANOVAs including all groups enabled pairwise ANOVAs between groups.

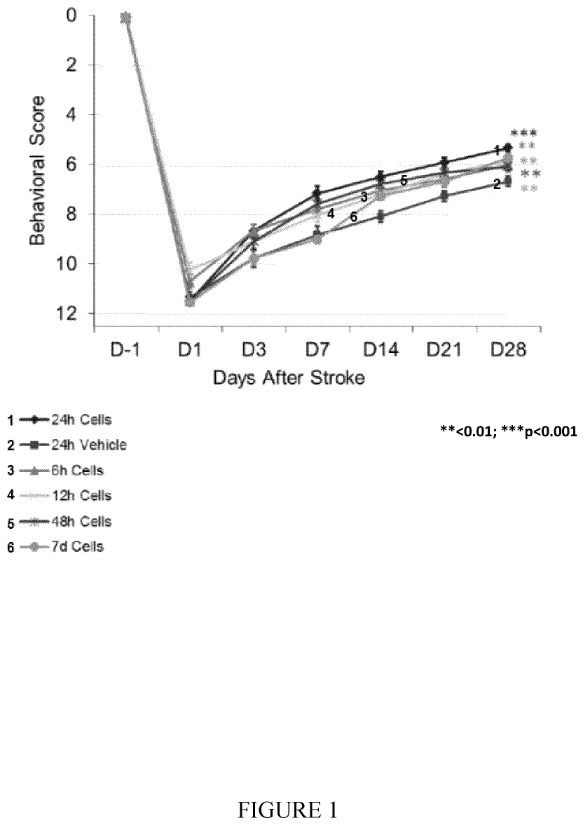

In FIGS. 1-3: *=different from vehicle-treated group at p<0.05;
**=different from vehicle-treated group at p<0.01;
***=different from vehicle-treated group at p<0.001
For the behavioral tests, the day before stroke, day −1, was purposely excluded from the analysis to ensure normal distribution of the data.

### Results

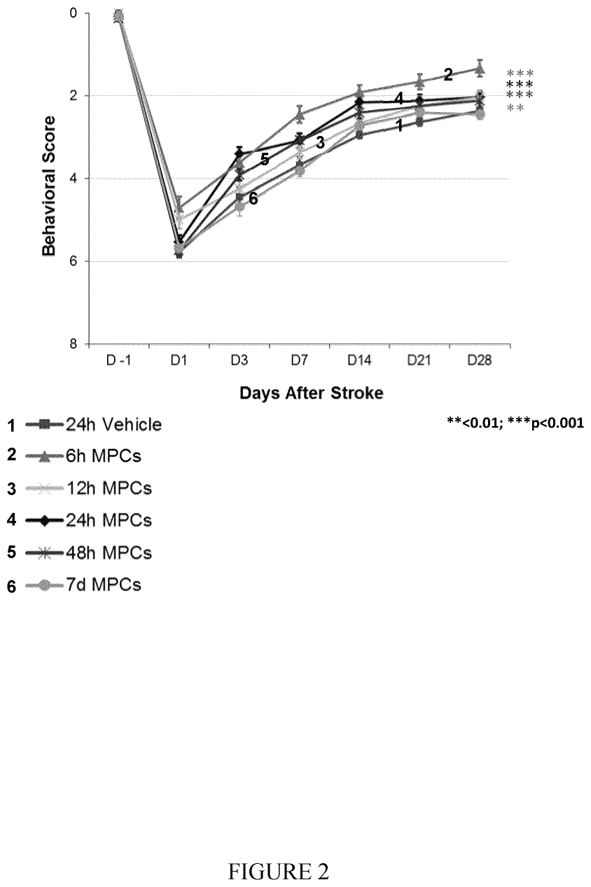

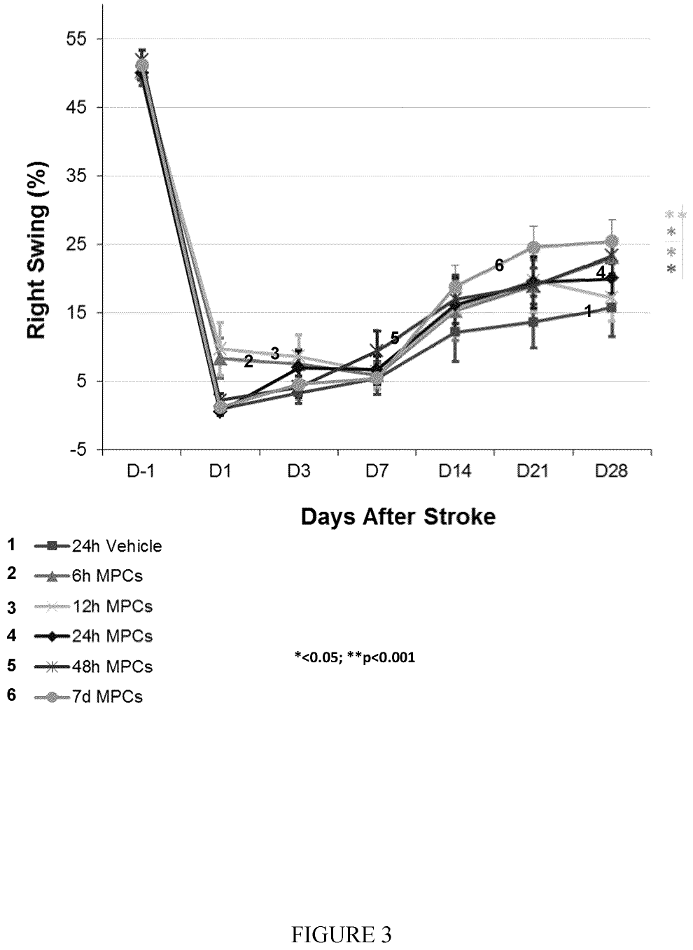

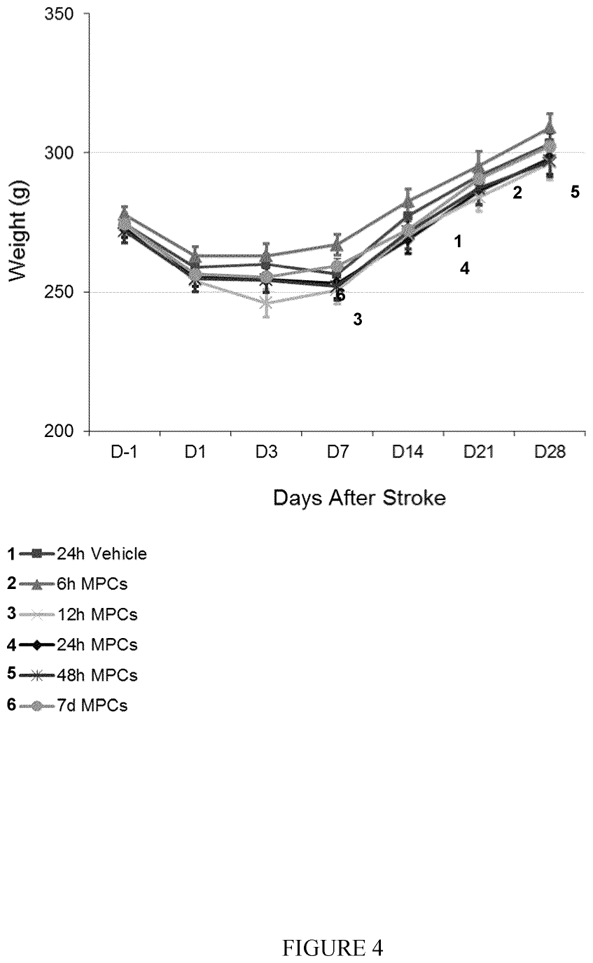

As shown in FIG. 1, administration of hMPCs at 6 hours (p<0.01), 12 hours (p<0.01), 24 hours (p<0.001), 48 hours (p<0.01), and 7 days (p<0.01) post MCAO significantly improved forelimb recovery compared to animals receiving vehicle administration. As shown in FIG. 2, administration of hMPCs at 6 hours (p<0.001), 12 hours (p<0.01), 24 hours (p<0.001), and 48 hours (p<0.001) post MCAO significantly improved hindlimb recovery compared to vehicle administration. Administration of hMPCs at 6 hours (p<0.05), 12 hours (p<0.05), 48 hours (p<0.01), and 7 days (p<0.01) post MCAO significantly improved body swing recovery compared to vehicle administration (FIG. 3). There were no significant differences in body weight between the MPC treated groups and vehicle groups (FIG. 4).

### Example 2

### Effects of Intravenous hMPCs or Vehicle on Functional Imaging in a Rat Model of Cerebral Infarct

### Infarct Model

Animals were anesthetized in an induction chamber with 2-3% isoflurane in N20:02 (2:1) and maintained with 1-1.5% isoflurane via face mask. Once anesthetized, animals received cefazolin sodium (40 mg/kg, i.p.) and buprenorphine (0.1 mg/kg, s.c.). A veterinary ophthalmic ointment, Lacrilube was applied to the eyes to keep them from drying. All animals were maintained at 37.0±1° C. during the surgical procedure. A small focal stroke (infarct) was made on the right side of the surface of the brain (cerebral cortex) by middle cerebral artery occlusion (MCAO) in all animals. Using aseptic procedures, an incision was made midway between the eye and eardrum canal. The temporalis muscle was isolated, bisected, and reflected.

A small window of bone was removed via drill and rongeurs (subtemporal craniectomy) to expose the MCA. Using a dissecting microscope, the dura was incised, and the MCA permanently occluded electrocoagulating from just proximal to the olfactory tract to the inferior cerebral vein (taking care not to rupture this vein), using microbipolar electrocauterization. The MCA was then transected. The temporalis muscle was then repositioned, and the incision was closed subcutaneously with sutures. The skin incision was closed with surgical staples (2-3 required). After surgery, animals remained on a heating pad until they recovered from anesthesia. They were then returned to clean home cages. They were observed frequently on the day of MCAO surgery (Day 0) and at least once daily thereafter until transferred to Ekam Imaging, Inc. for imaging on Day 8. All imaging analyses were completed blinded without any information as to treatment. Upon completion of the image analysis, the code was unblinded.

Imaging Protocol—Functional Magnetic Resonance Imaging (fMRI)

Rowlett Nude (RNU) rats were obtained from Taconic. Health certificates for animals were provided at the time of transportation on Day 8. Animals were transported to an imaging facility in a climate-controlled vehicle on each day of imaging (Day 15). A total of two groups, blinded as A and B (n=9/group) were studied. In addition, two animals not subjected to surgery were included and imaged on the first imaging day and used as normal controls for comparison.

Imaging studies were conducted using a Bruker Biospec 7.0T/20-cm USR horizontal magnet (Bruker, Billerica, Mass. U. S.A) and a 20-G/cm magnetic field gradient insert (ID=12 cm) capable of a 120-μs rise time (Bruker). Radiofrequency signals were sent and received with the quad coil electronics built into the animal restrainer. All animals were anesthetized and placed in the restrainer and imaged, acquiring the following anatomical and functional scans.

1) A pilot scan (RARE Tripilot)

2) T2 weighted reference anatomy scan of whole brain. (22 slices; 1.2 mm; FOV 3cm²; 256×256; RARE pulse sequence.)

3) fMRI (96×96×22, T2 weighted Rapid Acquisition with Refocused Echoes (RARE) images; foot shock by electrical stimulus, 0.6 mA, 3 minute baseline followed by three minute stimulus on LEFT hind paw, followed by another 3 minutes of baseline and then three minute stimulus on LEFT forepaw. That is, stimulation was applied to the paws contralateral to the stroke (and the matching paws on the controls).

4) fMRI (96×96×22, T2 weighted RARE images; 10% CO₂ Challenge).

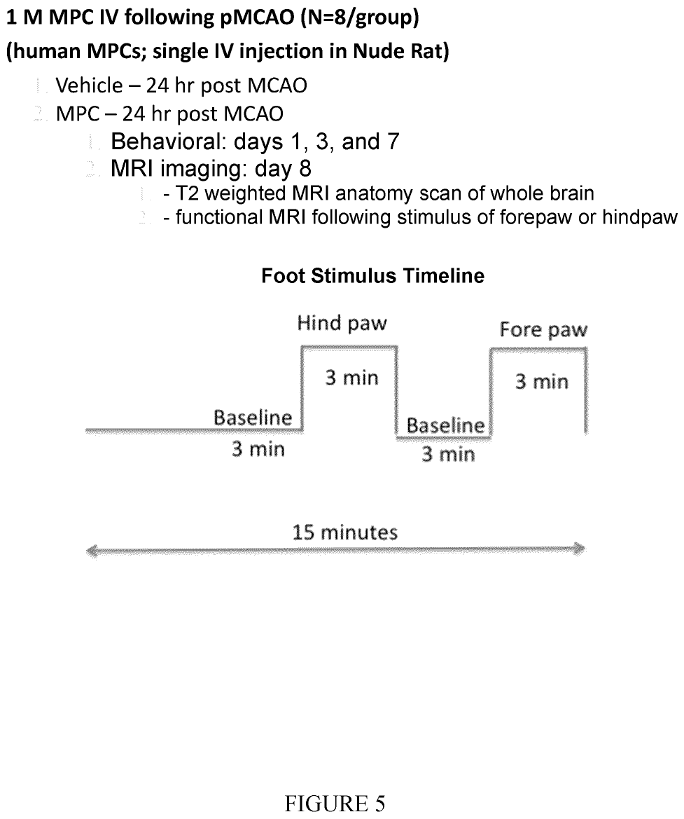

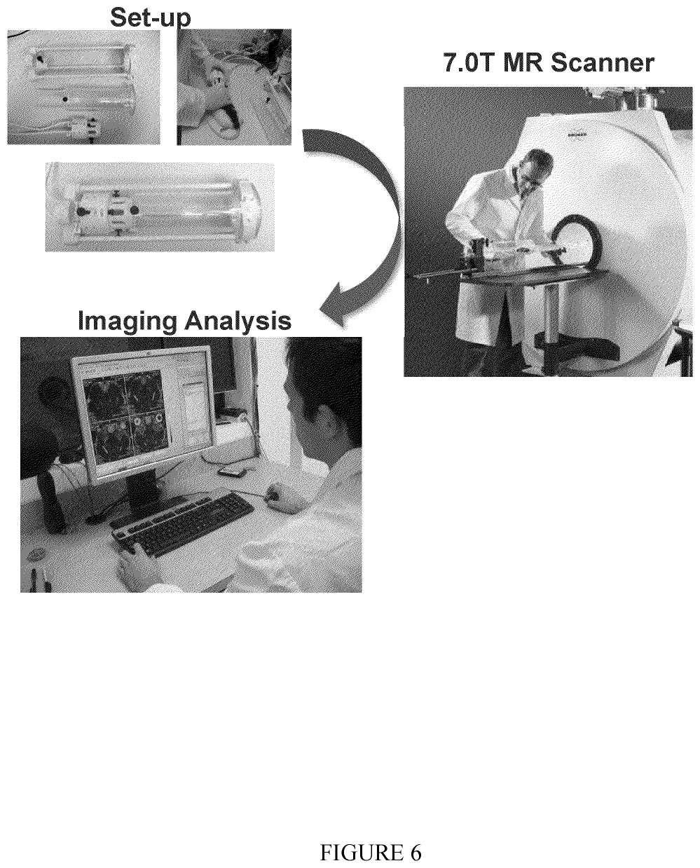

A schematic overview of the imaging experimental protocol and setup is shown in FIGS. 5 and 6. Respiration was monitored during imaging using a multi-animal monitoring and gating systems (SAII, Stony Brook, N.Y.).

### Image Analysis

The study consisted of six different MR Imaging modalities and hence various software and platforms were used to analyze the data. Data were compiled in the document format consisting of figures/Images and tables where numbers were reported. Functional MR images (fMRI) were analyzed using in house software MIVA where each subject was registered to a segmented rat brain atlas (Ekam Imaging). The alignment process was facilitated by an interactive graphic user interface. The composite statistics were built using the inverse transformation matrices. Each composite pixel location (i.e., row, column, and slice), premultiplied by [Ti]⁻¹, mapped it within a voxel of subject (i). A tri-linear interpolation of the subject's voxel values (percentage change) determined the statistical contribution of subject (i) to the composite (row, column, and slice) location. The use of [Ti]⁻ ensured that the full volume set of the composite was populated with subject contributions. The average value from all subjects within the group was used to determine the composite value. The average number of activated pixels that had highest composite percent change values in a particular ROI was displayed in a composite map. Activated composite pixels were calculated as follows:

$$Activated Composite Pixels ROI ( j ) = \frac{{\sum }_{i = 1}^{N} Activated Pixels Subject ( i ) ROI ( j )}{N}$$

The composite percent change for the time history graphs for each region was based on the weighted average of each subject, as follows:

$$Composite Percent Change = \frac{{\sum }_{i = 1}^{N} Activated Pixel Subject ( i ) \times Percent Change ( i )}{Activated Composite Pixels}$$

where N is number of subjects.

### Tissue Sample Collection

On day 8 post-MCAO, following imaging, rats were anesthetized deeply with CO₂. The heart was exposed and 18G needle inserted and connected through an infusion pump into the left ventricle toward the top where the ascending aorta connects, while the heart was still beating. A cut at the atrium was made to allow blood/perfusion solution to flow out. The perfusion was started with saline with approximately 2 Units/mL heparin at a rate of 40 mL/min for five minutes, and then 10% formalin at the same rate for five minutes. Following decapitation, the brain was carefully removed and placed into a labeled tube containing at least 10 mL of 10% formalin.

### Results

### Infarct Volume

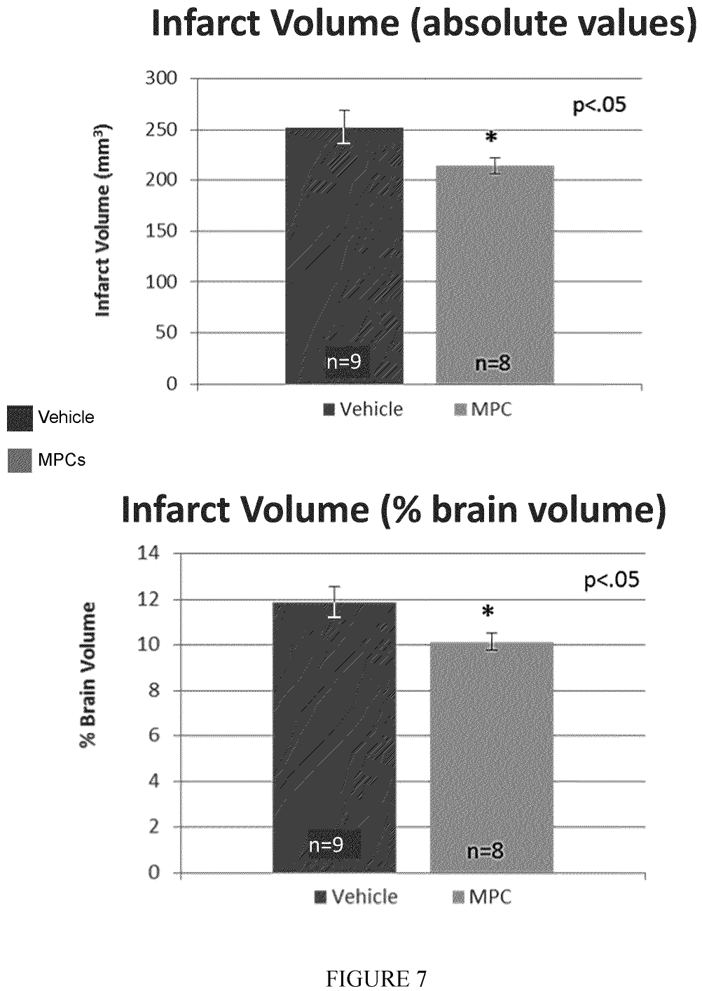

FIG. 7 shows measurements (mean±SEM) at time of MRI on day 8 for the infarct volume (top graph), and the infarct volume as a percent of whole brain (bottom graph). The MPC treated group had statistically smaller infarct volume compared to vehicle treated group (p<0.05). Additionally, the infarct volume as a percent of whole brain was significantly smaller in the MPC treated group (p<0.05).

### Cortical Activation Due to Paw Stimulation

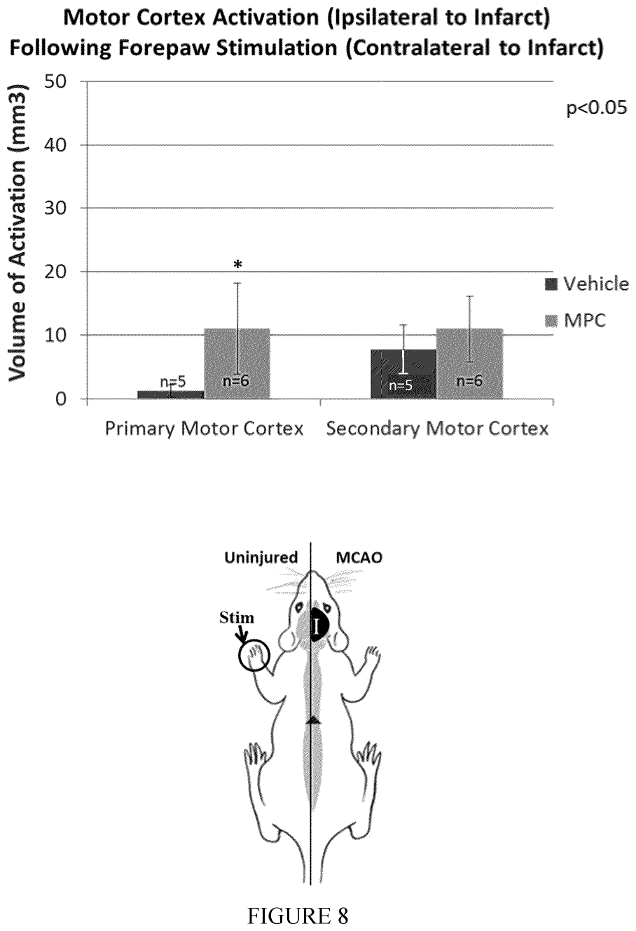

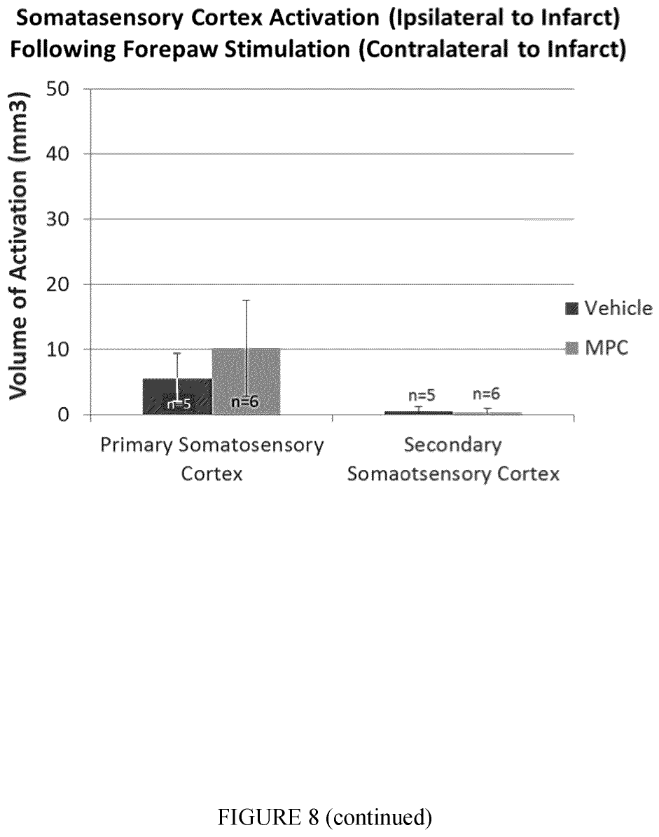

FIG. 8 shows activation shown by BOLD imaging due to forepaw stimulus contralateral to the infarct in vehicle and hMPC-treated groups in primary and secondary motor cortex and primary and secondary somatosensory cortex ipsilateral to the infarct. As shown in FIG. 8 (top panel), there was a significantly higher volume of activation in the primary cortex (but not secondary motor cortex) of the hMPC-treated group. No significant difference between the groups was shown for activation of somatosensory cortex (bottom panel). Likewise, no difference in activation of motor or somatosensory cortex contralateral to the infarct were observed (data not shown).

fMRI Analysis of Neuronal Activity in the Ischemic Core and Penumbra

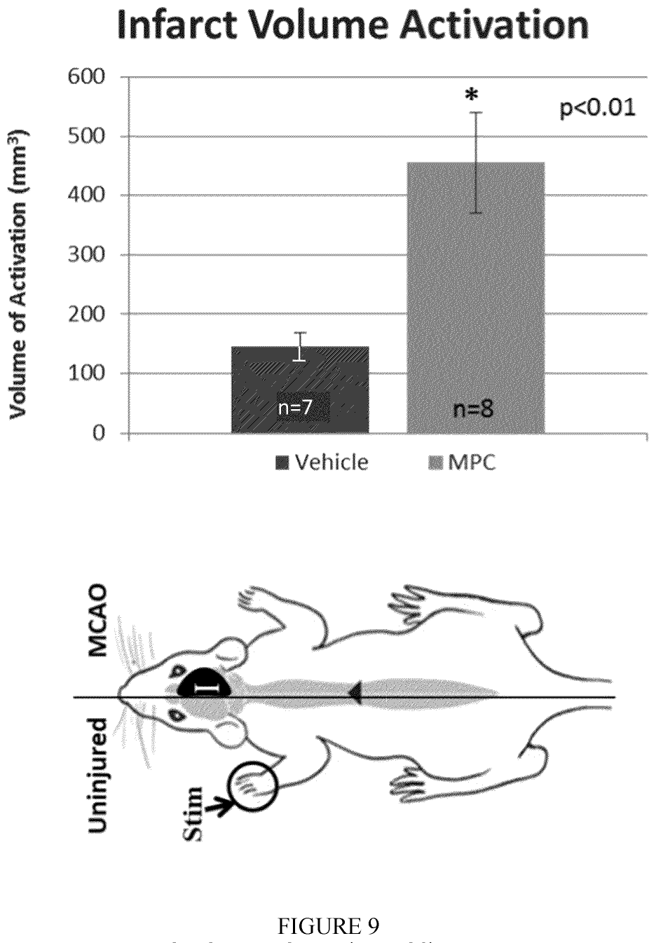

For post-hoc image analysis, an anatomical scan was used to identify the ischemic area. The fMRI activation was measured following forepaw stimulation contralateral to the infarct. The volume of activation was significantly greater in the MPC-treated animals (FIG. 9).

1. A method for increasing cortical activation or reducing infarct volume following a cerebral infarction, the method comprising systemic administration of a therapeutically effective amount of a human cell population enriched for mesenchymal lineage precursor or stem cells (MLPSCs) to a human subject in need thereof.

2. The method according to claim 1, wherein the cerebral infarction is an ischemic cerebral infarction.

3. The method according to claim 2, wherein the cerebral infarction was caused by hypoxic ischemic encephalopathy (HIE).

4. The method according to claim 1, wherein the cerebral infarction is a hemorrhagic cerebral infarction.

5. The method according to any one of claims 1 to 4, wherein the cerebral infarction is in motor cortex.

6. The method according to any one of claims 1 to 5, wherein the affected volume is reduced following the administration.

7. The method according to any one of claims 1 to 6, wherein the cortical activation is increased following the administration.

8. The method according to any one of claims 1 to 7, wherein the cortical activation is increased within the volume of the infarct.

9. The method according to any one of claims 1 to 8, wherein motor function is improved in the human subject following the administration.

10. The method according to any one of claims 1 to 9, wherein the increase in cortical activation is in response to contralateral tactile stimulation.

11. The method according to any one of claims 1 to 10, wherein the systemic administration is performed at about 24 hours or less following the cerebral infarction.

12. The method according to any one of claims 1 to 10, wherein the systemic administration is performed at about 12 hours or less following the cerebral infarction.

13. The method according to any one of claims 1 to 12, wherein the MLPSCs are STRO-1⁺ MPCs.

14. The method according to claim 13, wherein the STRO-1⁺ MPCs are STRO-1^{bright} MPCs.

15. The method according to any one of claims 1 to 14, wherein the STRO-1⁺ MPCs are tissue non-specific alkaline phosphatase (TNAP)⁺ or CD146⁺.

16. The method according to any one of claims 1 to 12, wherein the MLPSCs are mesenchymal stem cells.

17. The method according to any one of claims 1 to 16, wherein the human cell population is an allogeneic human cell population.

18. The method according to any one of claims 1 to 16, wherein the human cell population is an autogeneic human cell population.

19. The method according to any one of claims 1 to 18, comprising administering about 2×10⁶ cells/cm³ of affected cortex to about 2×10⁷ cells/cm³ of affected cortex.

20. The method according to any one of claims 1 to 18, comprising administering 0.1×10⁶ cells/kg body weight to 5×10⁶ cells/kg body weight.

21. The method according to any one of claims 1 to 20, wherein the human cell population was culture expanded prior to the administration.

22. The method according to any one of claims 1 to 21, wherein the human cell population was derived from bone marrow, dental pulp, adipose, or pluripotent stem cells.

23. The method according to any one of claims 1 to 21, wherein the human cell population was not derived from dental pulp or adipose.

24. The method according to any one of claims 1 to 23, wherein the human cell population is a genetically modified human cell population.

25. The method according to any one of claims 1 to 24, wherein the systemic administration is intra-arterial administration or intravenous administration.

26. The method according to claim 25, wherein the systemic administration is intra-arterial administration.

27. The method according to any one of claims 1 to 26, further comprising administering a thrombolytic agent.

28. The method according to any one of claims 1 to 26, wherein the subject is not administered a thrombolytic agent before or after administration of the human cell population.

29. The method according to any one of claims 1 to 28, further comprising administering mannitol.

30. The method according to claim 29, further comprising administering temozolomide.

31. The method according to any one of claims 1 to 30, further comprising administering an anti-inflammatory agent.

32. The method according to any one of claims 1 to 31, wherein the human cell population is administered a plurality of times.

33. The method according to any one of claims 1 to 32, wherein the human cell population is administered once every four or more weeks.

34. The method according to any one of claims 1 to 31, wherein the human cell population is administered a single time.

35. The method according to any one of claims 1 to 34, wherein at least a portion of the cells in the human cell population is labelled for in vivo detection.

36. The method according to claim 35, further comprising tracking the location of the labelled cells in the subject following the administration.

37. The method according to any one of claims 1 to 36, further comprising determining changes in infarct volume and/or activity within the infarct volume following the administration.
